(19) United States
(12) Patent Application Publication
(10) Pub. No.: US 2020/0002482 A1
(43) Pub. Date: Jan. 2, 2020

(54) METHOD FOR TREATING LIGNOCELLULOSIC BIOMASS

(71) Applicants: Institut National De La Recherche Agronomique, Paris Cedex (FR); IFP Energies Nouvelles, Rueil-Malmaison Cedex (FR); Agro Industries Recherche Et Developpement, Pomacle (FR)

(72) Inventors: Caroline AYMARD, Rueil-Malmaison Cedex (FR); Romain ROUSSET, Rueil-Malmaison Cedex (FR); Larissa PEROTTA, Lyon (FR); Emilia KNOSPE, Lyon (FR)

(73) Assignees: Institut National De La Recherche Agronomique, Paris Cedex (FR); IFP Energies Nouvelles, Rueil-Malmaison Cedex (FR); Agro Industries Recherche Et Developpement, Pomacle (FR)

(21) Appl. No.: 16/451,946

(22) Filed: Jun. 25, 2019

(30) Foreign Application Priority Data
Jun. 27, 2018 (FR) 1855789

(51) Int. Cl. C08H 8/00; C12P 7/10; C12P 7/16; C12P 7/36

(52) U.S. Cl. CPC C08H 8/00; C12P 7/10; C12P 2201/00; C12P 7/36; C12P 7/16

(57) ABSTRACT

The invention relates to a process for treating a lignocellulosic biomass, with:
a. Pretreatment of the biomass via at least one cooking or steam explosion operation, so as to obtain a pretreated substrate,
b. Liquid/solid separation on at least a portion of the pretreated substrate, comprising two successive substeps:
an upstream substep b1 of contacting the solid/liquid mixture performed with a continuous mixer (M) using a mixing fluid,
a downstream sub-step b2 of extraction/washing performed with a continuous filter, using a washing fluid, to obtain a solid phase enriched in solid and a plurality of liquid phases enriched in liquid, with at least partial recycling of a liquid phase extracted from the filter at the inlet of the mixer as a mixing fluid.

### FIELD OF THE INVENTION

The invention relates to a process for treating lignocellulosic biomass for producing “second-generation” (2G) sugary liquors. These sugary liquors may be used to produce other products via a chemical or biochemical/fermentation pathway (e.g. alcohols such as ethanol, butanol or other molecules, for example solvents such as acetone and other biobased molecules, etc.).

### PRIOR ART

Lignocellulosic biomass represents one of the most abundant renewable resources on Earth. The substrates considered are very varied, and concern both ligneous substrates such as various woods (broad-leaved and coniferous), byproducts derived from agriculture (wheat straw, rice straw, corn husks, etc.) or from other agrifood, papermaking, etc. industries. Lignocellulosic biomass is composed of three main polymers: cellulose (35% to 50%), which is a polysaccharide consisting essentially of hexoses; hemicellulose (20% to 30%), which is a polysaccharide consisting essentially of pentoses; and lignin (15% to 25%), which is a polymer of complex structure and of high molecular weight, composed of aromatic alcohols linked via ether bonds. These various molecules are responsible for the intrinsic properties of the plant wall and organize themselves into a complex entanglement. Among the three base polymers that make up the lignocellulosic biomass, cellulose and hemicellulose are the ones that enable the production of 2G sugary liquors.

The process for the biochemical conversion of the lignocellulosic material into 2G sugary liquors comprises notably a pretreatment step and a step of enzymatic hydrolysis with an enzymatic cocktail. These processes also usually include an impregnation step before the pretreatment. The sugary liquors derived from the hydrolysis may be used/upgraded as they are or optionally subsequently processed, for example in a fermentation or chemical process. Usually, the overall process comprises intermediate separation steps and/or a step of purification of the final product.

The pretreatment makes it possible to modify the physicochemical properties of the lignocellulosic biomass so as to make the cellulose accessible to the enzymes and to achieve good reactivity in enzymatic hydrolysis. Many pretreatment techniques exist and allow establishment of the temperature of the biomass under varied chemical conditions. The pretreatment may be performed with or without addition of acidic or basic products. It may also be performed in a solvent such as water or an organic product, for instance alcohol (organosolv process), but also in a sparingly diluted medium such as steam. This pretreatment may also involve a physical step such as defiberizing or explosive decompression in the context of a steam explosion. This pretreatment may also involve several steps for optimizing the overall process, for instance acidic cooking followed by a steam explosion or two consecutive steam explosions. The pretreatments below grouped under the generic term “cooking” concern treatments of biomass under diluted conditions, and include acidic cooking, alkaline cooking and “organosolv” cooking. The latter process concerns a pretreatment in the presence of one or more organic solvents and generally water. The solvent may be an alcohol (ethanol), an acid such as acetic acid or formic acid, or else acetone. “Organosolv pulping” processes lead to at least partial dissolution of the lignin and partial dissolution of the hemicelluloses. There are thus two outlet streams: the pretreated substrate with residual cellulose, hemicellulose and lignin, and the solvent phase which contains the dissolved lignin and a portion of the hemicelluloses. There is generally a step of regeneration of the solvent which makes it possible to extract a lignin stream. Certain “organosolv pulping” treatments (notably with ethanol) are coupled with the addition of a strong acid (such as H₂SO₄). It may also be envisaged to place the biomass in contact with the solvent via an impregnation reactor before the cooking phase or to place the biomass in contact with the acid catalyst before performing “organosolv pulping” cooking.

The pretreatment of steam explosion type is different from a treatment of cooking type in the sense that the biomass is concentrated, and subjected to a proportionately small amount of steam. Usually, steam explosion under acidic conditions is preferred, since it allows a good compromise between acidic hydrolysis of the hemicellulose and the reactivity of cellulose in enzymatic hydrolysis, with virtually total hydrolysis of the hemicellulose and a large improvement in the availability and reactivity of the cellulose to the enzymes. This pretreatment may be preceded by other treatment(s) (milling, impregnation, cooking, etc.).

Various configurations are reported, for example, in the publication “Production of bioethanol from lignocellulosic materials via the biochemical pathway: a review”, M. Balat, Energy Conversion and Management 52 (2011) 858-875, or in the publication “Bioethanol production from agricultural wastes: an overview”, N. Sarkar, S. Kumar Ghosh, S. Bannerjee, K. Aikat, Renewable Energy 37 (2012) 19-27.

It is thus noted that this type of process, cooking or steam explosion, requires a transformation of the raw biomass into a reactive pretreated substrate (also known as pretreated marc), before starting the subsequent conversion steps per se. After this pretreatment, sugars (C5 and C6 sugars) are found in a sugary liquor impregnating the pretreated marc. The recovery of this juice may be advantageous for upgrading in the rest of the process of biomass transformation or in another process (in parallel) or for marketing as sugary liquor. It is thus advantageous to extract these pretreated marc liquors, before the pretreated marc in question, which will then be enriched in solid matter, undergoes the subsequent treatments.

The pretreatment step is often preceded by an impregnation step. It is followed by a step of enzymatic hydrolysis using an enzymatic cocktail. In certain cases, these preceding steps are followed by a step of ethanolic fermentation of the sugars released and a step of purification of the fermentation products. In certain configurations of the process scheme, the steps of enzymatic hydrolysis and of fermentation may take place in the same reactor, in a fermentation configuration known as SSF (Simultaneous Saccharification and Fermentation). When these two steps of the process are separated, such a scheme is referred to as being of the SHF type (Separated Hydrolysis and Fermentation). Examples are given in the document “Ethanol from lignocellulosics: A review of the economy”, M. von Silvers and G. Zacchi, Bioresource Technology 56 (1996) 131-140.

In these steps also, there may be products in the form of a solid/liquid mixture, for example “hydrolysate” products obtained after the enzymatic hydrolysis, where it may be advantageous to perform a solid/liquid separation, to extract the liquid phase, for example to use the liquors produced in a subsequent step that is sensitive to the presence of solid, for example in the case of sensitivity of the fermentation microorganism used in a subsequent fermentation step, or in the case of recycling of the microorganism around this fermentation step.

It is thus seen that at various steps in the biomass treatment processes, the products being treated are in the form of solid/liquid mixtures, and that it may be advantageous to extract at least a portion of the liquid phase from the solid phase in order to upgrade it and to optimize the overall process.

Patent WO 2014/135755 proposes a process for treating biomass via the following succession of steps: a—a pretreatment step is performed by placing in contact and heating the biomass feedstock with water and an acidic or basic compound, so as to obtain a pretreated substrate, b—the pretreated substrate is placed in contact with cellulase enzymes and a liquid stream enriched in fermentation products obtained in step e) so as to obtain a hydrolysate including a solid residue and a liquid phase containing sugars, c—alcoholic fermentation of the hydrolysate is performed using an alcohol-forming microorganism so as to produce a fermentation wine including a solid material and a liquid phase containing fermentation products, d—at least a portion of the solid material contained in the fermentation wine is extracted so as to obtain a stream enriched in solid material and a fermentation wine depleted in solid material, e—the stream enriched in solid material is washed with a liquid stream so as to obtain said liquid stream enriched in fermentation products, the liquid stream enriched in fermentation products being recycled into step b), f—a step of separation of the fermentation wine depleted in solid material is performed so as to obtain at least one purified stream including an alcohol or a solvent and at least one stream of vinasse.

Thus this patent proposes to perform the separation of lignin and of other possible inert solids after the fermentation step. The solid material predominantly composed of lignin is then subjected to washing to recover the trapped fermentation products, in particular the alcohols and the solvents. The washing liquid is then recycled into the enzymatic hydrolysis unit, which may be the same unit as the fermentation unit or which may be different from the fermentation unit so as not to bring about dilution in the existing streams.

Patent EP 2 774 992 proposes to perform, in a process including a pretreatment step, a step of enzymatic hydrolysis and then a fermentation step, to extract at least a portion of the solid residue contained in the hydrolysate so as to obtain a stream of solid residue including lignin and a hydrolysate depleted in solid residue, and the stream of solid residue is then washed with a liquid stream so as to recover a liquid stream enriched in sugars, which may be recycled into the enzymatic hydrolysis step to be able to upgrade the sugars without bringing about dilution of the streams in the process.

In these two patents, washes and extractions of intermediate products are performed, directed towards separating out from a solid/liquid mixture a phase enriched in solid material and a phase enriched in liquid, using conventional devices such as a decantation or percolation device to perform the extraction/separation and a counter-current washing device.

However, achieving such a solid/liquid separation is a difficult operation to standardize, in so far as the mixture may have very different characteristics at the same time, notably depending on its place in the sequence of steps of the overall biomass treatment process, depending on the type of biomass used, etc. These characteristics are notably the solids content of the initial biomass or the rheology of the mixture to be treated. The aim of the separation may also differ, depending on the point up to which it is desired to extend the separation. Conventional devices do not necessarily have all the versatility and efficiency required for ensuring separations on such different mixtures.

The aim of the invention is then to conceive of a novel way of performing solid/liquid separations starting with a mixture, which is improved when compared with the conventional techniques, notably in the context of biomass treatment processes. The aim of the invention is notably to conceive of techniques that are more efficient and/or more versatile depending on the mixture to be treated.

Optionally, the aim of the invention is to conceive of this type of technique which, in addition, may be performed with devices that are as compact as possible.

### SUMMARY OF THE INVENTION

The subject of the invention is firstly a process for treating a lignocellulosic biomass, said process comprising at least the following step:
a. Pretreatment of the biomass via at least one cooking or steam explosion operation, so as to obtain a pretreated substrate.

This process also comprises at least one step of:
b. Liquid/solid separation on at least a portion of the pretreated substrate obtained on conclusion of step a or on conclusion of an additional step of treatment of said substrate after step a, said separation step comprising two successive substeps:
an upstream substep b1 of contacting the solid/liquid mixture performed with a continuous mixer using a mixing fluid, optionally preceded by a pump, and
a downstream sub-step b2 of extraction/washing performed with a continuous filter, notably a belt filter, using a washing fluid, to obtain a solid phase enriched in solid and a plurality of liquid phases enriched in liquid, with the filter preferably functioning counter-currentwise between the circulation of the solid/liquid mixture to be separated and the extraction/washing fluid, and at least partial recycling of a liquid phase extracted from the belt filter at the inlet of the mixer as a mixing fluid.

Throughout the present text, the terms “upstream”, “downstream”, “following” or “preceding” are given with reference to the general direction of flow of the product in question to be treated, to be more precise of the solid part of the biomass to be treated, in the installation in which the process of the invention is performed.

To perform the solid/liquid separation, the invention thus proposes to couple two operations with two devices specific to each of them, namely, first a mixer for placing in contact the mixture to be separated with a mixing fluid (for example an aqueous fluid), which can function continuously, and which then feeds a belt filter to perform the actual separation. The mixer gives the mixture the appropriate characteristics (rheological properties, solids content, etc.) to be able then to be treated by the continuous filter. The two devices may be mounted in series, both functioning continuously, which is markedly more advantageous than batchwise functioning. For the filter, the term “continuous” is understood for the solid to be treated.

The continuous mixer is known in its principle: it is, schematically, a mixer comprising a hollow cylindrical body with an inlet fed with the mixture, an internal screw for conveying the mixture from the inlet to the outlet of the mixer, and a mixing fluid (water) circuit placing the mixture in the mixer in contact with the fluid in question. The mixer may be equipped at the inlet with a metering device. This type of continuous mixer is notably sold by the company Parimix under the name Parimix IMR Continuous Mixer. It notably has the advantage of being able to place in contact a mixture, even at high flow rate, with a low mixing chamber volume, which makes the device very compact, which is most particularly sought in the context of the invention. It also allows a short residence time of the mixture in the mixer, while at the same time making it possible to obtain at the mixer outlet a product that is very homogeneous and at the desired solids content.

The preferred continuous filter is a belt filter, which is also known in its principle: schematically, it makes it possible to perform the extraction and washing of a solid/liquid mixture continuously, by conveying the mixture successively on different zones of a gauze belt. The extraction is performed under partial vacuum through this gauze which transports the mixture, from zone to zone, and which is porous to be able to perform filtration/extraction. It may be equipped, in its most downstream part, with a press to complete the extraction of the liquid. This type of belt filter is marketed, for example, by the company BHS under the name BFR Continuous Belt Filter. This type of tooling is known as a continuous belt filter with vacuum extraction.

Other continuous filters may also be used, for example in the form of a belt press or in the form of a plurality of belt presses in series.

The invention thus uses in series these two devices both functioning continuously (the belt filter functioning, however, sequentially in the strict sense, as shall be detailed later), the mixer outlet feeding the inlet of the filter (which is markedly more advantageous than batchwise functioning), resulting in an assembly that is very compact and easy to integrate into an existing biomass treatment installation.

The thing which the invention has discovered is that the combination of two choices of functioning parameters of these devices was the necessary condition in order for the separation obtained first to be industrially feasible, and for it also to be robust and efficient:
on the one hand, the choice of continuous functioning of a filter, notably of a belt filter and preferably functioning counter-currentwise between the circulation of the solid/liquid mixture to be separated and the extraction/washing fluid (counter-currentwise functioning proving to be more efficient than co-currentwise functioning),
on the other hand, at least partial recycling of a liquid phase extracted from the filter at the mixer inlet as mixing fluid, which made it possible to reduce the consumption of mixing fluid, generally of water, of the mixer, and which proved, surprisingly, to allow more efficient mixing in the mixer and extraction of more concentrated liquid phase(s) on the filter.

Preferably, the separation of the solid/liquid mixture is performed during step b on a first portion of the pretreated substrate obtained on conclusion of step a, and a second portion of said pretreated substrate is subjected to at least one subsequent treatment step, notably hydrolysis, and then fermentation, and the solid phase enriched in solid obtained in step b is also subjected to at least one subsequent treatment step, notably the same as those to which the second portion of the pretreated substrate is subjected.

It is thus possible to extract a portion of the liquid of the pretreated marc, all of the marc simply pretreated and of the marc pretreated and then separated according to the invention then following the same sequence of steps, and being injected, for example, into the same reactor of the following step to undergo the same transformation therein.

The advantage is then that of being able to take up just a portion of the liquid phase (just the sufficient amount) on a portion of the pretreated marc, for the purpose of upgrading or exploitation in the very process of the liquid phase containing sugars (also referred to as “sugary liquor” in the present text).

Advantageously, according to one embodiment, the liquid phases enriched in liquid derived from the separation step b are sugary liquors, and said process also comprises a step c of producing enzymes and/or a step d of producing yeasts, and at least one of said sugary liquors is used for said production of enzymes c or of yeasts d (propagation/growth of microorganisms). It is thus possible, in this case, to extract just the necessary amount of sugary liquor to feed the production of enzymes or of yeasts, and thus to limit the separation of the pretreated marc to the necessary amount.

The process according to the invention may advantageously comprise a step e of enzymatic hydrolysis of the pretreated substrate obtained from the pretreatment step a to obtain a hydrolysate, and the solid/liquid separation b may then be performed on at least a portion of said hydrolysate, notably on all of said hydrolysate.

According to one embodiment, the solid/liquid separation b is performed on at least a portion, notably all, of the pretreated substrate obtained on conclusion of the pretreatment step a to obtain a solid phase enriched in solid, and the process also comprises a step e of enzymatic hydrolysis of said solid phase enriched in solid to obtain a hydrolysate. In this case, it is thus preferred to separate out all of the pretreated marc and to exploit all of the extracted liquid phase, which is advantageous when the production of sugary liquor to upgrade it separately from the rest of the process is expressly targeted.

Advantageously, the process according to the invention also comprises the following steps after the pretreatment step a:
e. Enzymatic hydrolysis of the pretreated substrate, so as to obtain a hydrolysate including a solid material and a liquid phase containing sugars,
f. Fermentation of the hydrolysate, so as to obtain a fermentation wine, the fermentation step f possibly succeeding or being concomitant with the enzymatic hydrolysis step e,
g. Separation/Distillation of the fermentation wine, so as to obtain a distillation product in the form of an alcohol, a solvent or another biobased molecule, and vinasses,
   the separation step b being able to be performed on at least a portion, notably all, of the products obtained after the hydrolysis step e and/or fermentation step f and/or separation/distillation step g.

Optionally, in the separation step b, the interior of the mixer is heated to an operating temperature of at least 30° C., notably between 40 and 60° C., notably via integrated electrical heating means. In point of fact, it proved that heating of the mixture during its transportation inside the mixer improved the homogeneity of the mixture exiting the mixer. The temperatures remain low enough so as not to give rise to excessive energy consumption.

Preferably also, in the separation step b, the extraction/washing fluid is heated before introduction into the filter and/or the extracted liquid phase from the filter is recycled before its introduction into the mixer, notably at a temperature of at least 30° C., and of not more than 90° C., notably a temperature of between 40° C. and 80° C. Heating the liquid phase before its recycling in the mixer enables said phase to participate in the effort of heating the mixture in the mixer. The heating obtained by the heating means with which the mixer is equipped and the heating obtained by circulation in the mixer of a hot mixing fluid can then be appropriately adjusted. Naturally, it is also possible to heat the mixing fluid not coming from recycling from the filter (generally water), in addition to or instead of heating of the liquid phase recycled from the filter.

Advantageously, in step b, the filter is a belt filter which comprises at least two, notably at least three, successive zones, with withdrawal in at least one zone, notably in each zone, of a liquid phase. The belt filters are of very flexible implementation: the number and size, notably the length of zones that is desired, may be envisaged, zones may be grouped together so as to withdraw only one liquid phase per group of zones, the zones may have different lengths, for example increasing or decreasing lengths along the axis of transportation of the mixture on the belt, etc.

Preferably, the liquid phase of the first and/or the second zone is at least partly recycled into the inlet of the mixer of step b. It is thus preferred to recycle the extracted liquid phase(s) as far upstream of the belt filter as possible, i.e. those that are the most concentrated, which makes it possible to obtain a more concentrated liquid at the mixer outlet. Preferably, the liquid phase withdrawn from the third zone and/or following zones and/or from the last zone is recycled at least partly as belt filter washing/extraction fluid: this is the way in which the counter-current is performed in the functioning of the belt filter in the invention.

Optionally, in step b, the filter, notably the belt filter, may be equipped with a press in its end part. The separation is thus further improved.

Advantageously, the operating parameters of the separation steps b1 and b2 may be selected as a function of the characteristics of the solid/liquid mixture, so that the liquid phases produced extracted from the belt filter have a concentration of at least 50 g of product/kg, notably at least 30, at least 35; at least 40, preferably at least 50 g of sugar/kg when the separation b is performed on the pretreated substrate obtained from the pre-treatment step a. It is in point of fact from such a concentration that the sugary liquors can effectively be upgraded as such.

The preferred operating parameters for achieving this result are in particular the rate of recycling of the liquid phase extracted from the filter at the inlet of the mixer, or else the configuration of recycling (namely a single type of recycled juice or several juices from several zones of the filter in particular, which will thus have different sugar concentrations, in adjustable proportions, and/or an addition of water in an amount adjustable to this or these recycled juices to the mixer)

A subject of the invention is also an installation for treating a lignocellulosic biomass, notably intended for performing the process described previously, and which comprises at least:
one zone for pretreatment of the biomass via at least one cooking or steam explosion operation, so as to obtain a pretreated substrate,
one zone for liquid/solid separation on at least a portion of the pretreated substrate obtained at the outlet of the pretreatment zone or of an additional treatment zone of said substrate after the pretreatment zone, said separation zone comprising two subzones in series: —one upstream contacting subzone comprising a continuous mixer using a mixing fluid, optionally associated with an upstream pump, and—one downstream extraction/washing subzone comprising a continuous filter, notably a belt filter with a washing fluid, the filter preferably functioning counter-currentwise between the circulation of the solid/liquid mixture to be separated and the extraction/washing fluid, and at least partial recycling of a liquid phase extracted by the belt filter into the inlet of the mixer as a mixing fluid.

The installation may provide, in the separation zone: —fluid connection means between the belt filter and the mixer to recycle at least a portion of a liquid phase extracted by the filter into the inlet of the mixer and—fluid connection means for recycling another liquid phase extracted from the filter as a washing fluid for said filter.

The filter is preferably a belt filter, which may optionally be equipped with a press in its end part.

The treatment installation may also comprise an enzyme production zone and/or a yeast production zone, and may envisage a means for transferring at least one liquid phase extracted from the belt filter from the separation zone to the enzyme production zone and/or to the yeast production zone, said liquid phase being a sugary liquor.

A subject of the invention is also the use of the process or of the installation described above for the treatment of biomass such as wood, straw, agricultural residues, and all dedicated energy crops, notably annual or perennial plants such as miscanthus in order to produce sugars, biofuels or biobased molecules. More generally, the lignocellulosic biomass or lignocellulosic materials employed in the process according to the invention is obtained, for example, from raw or processed wood (broad-leaved and coniferous), agricultural byproducts such as straw, plant fibre, forestry crops, residues from alcohol-generating, sugar-yielding and cereal-yielding plants, residues from the paper industry, marine biomass (for example cellulosic macroalgae) or cellulosic or lignocellulosic material transformation products. The lignocellulosic materials may also be biopolymers and are preferentially rich in cellulose.

The invention makes it possible to produce as upgradable products both biofuel such as ethanol and sugary liquors, or biofuel alone, or sugary liquors alone, with great flexibility.

### DETAILED DESCRIPTION

The invention will be described in detail below with the aid of non-limiting examples, illustrated by the following figures:

FIG. 1: a synoptic representation of the equipment used in the context of the invention for performing step b of separating a solid/liquid mixture in a biomass treatment process;

FIGS. 2a and 2b: two different exploitations of the equipment represented in FIG. 1 according to the type of biomass used;

FIG. 3: a schematic representation (longitudinal cross section) of the mixer pertaining to the equipment represented in FIG. 1;

FIG. 4: a functional representation in block diagram form of the separation step b performed by the invention incorporated into a biomass pretreatment step according to a first variant;

FIG. 5: a functional representation in block diagram form of a whole biomass treatment process incorporating the separation step b performed by the invention according to FIG. 4;

FIGS. 6,7,8: a functional representation in block diagram form of a whole biomass treatment process incorporating the separation step b performed by the invention according to, respectively, a second, third and fourth variant.

### DESCRIPTION OF THE FIGURES

The same references correspond to the same components/fluids/products on all of the figures. The figures are very schematic, are not necessarily to scale and do not represent all the components of the equipment or all the process details concerned, but only those which more particularly concern the description of the invention.

FIG. 1 thus represents the equipment developed in the context of the invention for performing the solid/liquid separation of a mixture in a lignocellulosic biomass treatment process: the mixture to be separated is first treated in a continuous mixer M such as an IMR mixer from Parimix, represented in greater detail in FIG. 3, and then with a belt filter F such as a BFR model continuous belt vacuum filter from BHS. They are both arranged substantially horizontally, at different heights, the mixer being arranged above the belt filter.

To simplify the description of this equipment, for the sake of brevity, it will be considered that the mixture to be separated is a pretreated marc from a biomass treatment process, although the separation according to the invention can be applied to other solid/liquid mixtures of such a process (as described later with the aid of FIG. 6 et seq.).

The mixer M allows repulping of the pretreated marcs with a high content of dry matter (DM) and of solids, by adding a mixing fluid and by blending obtained by the endless spiral of the mixer conveying the marc from one end to the other of the mixer. The mixer M, as detailed in FIG. 3, comprises a cylindrical body equipped with a shaftless rotating screw V, with its upstream end equipped with a metering device D which continuously measures out the mixture to be separated, which is then injected into the mixer via a feed tube or hopper A connected to the metering device. It is represented in the operating position, i.e. in a substantially horizontal plane.

In its upstream part, the cylindrical body of the mixer is equipped with mixing fluid inlet(s) e1, e2, the fluid(s) are centrifuged by the spirals, and the liquid vortex thus created meets the stream of exploded marc in the opposite direction. By means of this principle, the volume of the mixing chamber of this mixer is low, the power used is also low and the mixer as a whole is compact. Here, the mixer is equipped with electrical heating means (not shown) to heat the interior of the cylindrical body to a temperature of about 40 to 50° C., this supply of heat promoting the obtention of greater homogeneity of the mixture leaving the mixer at a constant residence time therein. The repulped marc leaving the mixer M falls by gravity onto the upstream part of the belt of the belt filter F. Separation of this marc on the belt filter F with counter-current washing makes it possible to extract liquors concentrated in sugars. The belt filter F technology is based on vacuum filtration, the principle of which is to sequentially spread the pretreated marc over an advancing belt. This porous belt makes it possible, by vacuum suction, to separate the liquids (the liquors) from the solid, forming a cake (the washed marc), which falls at the end of the belt. Washing fluid is sprayed over the belt to wash the cake which gradually forms along the belt. The liquors are partially recycled, as detailed later. They may also be pooled as a single stream.

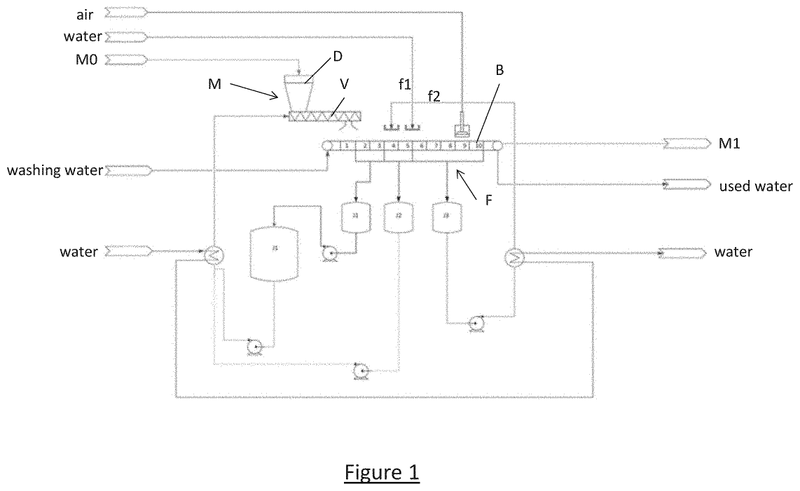

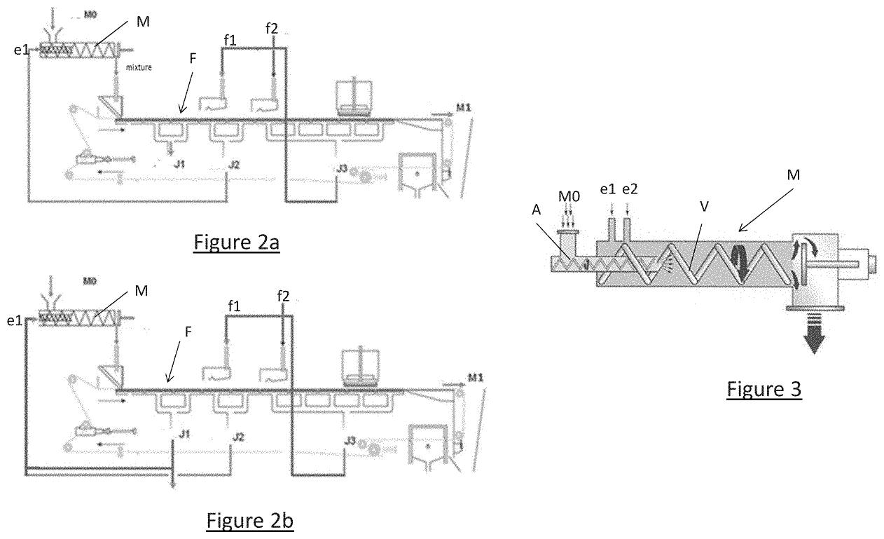

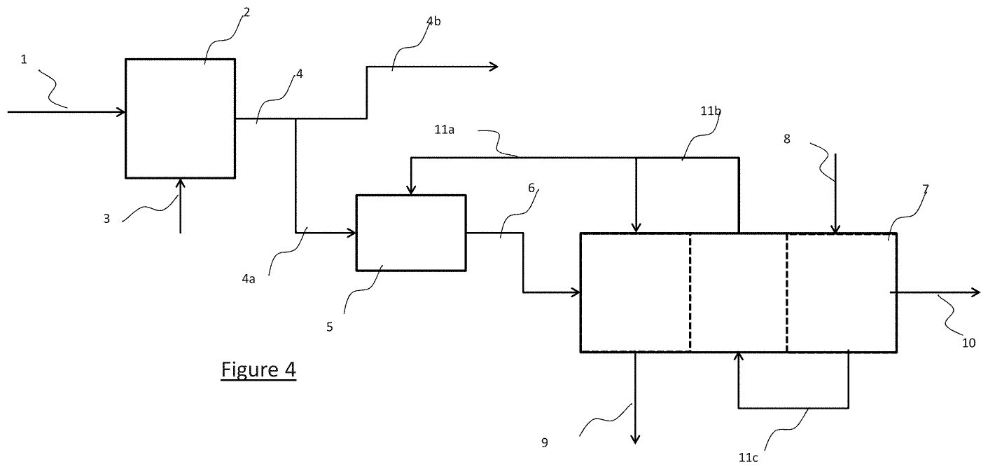

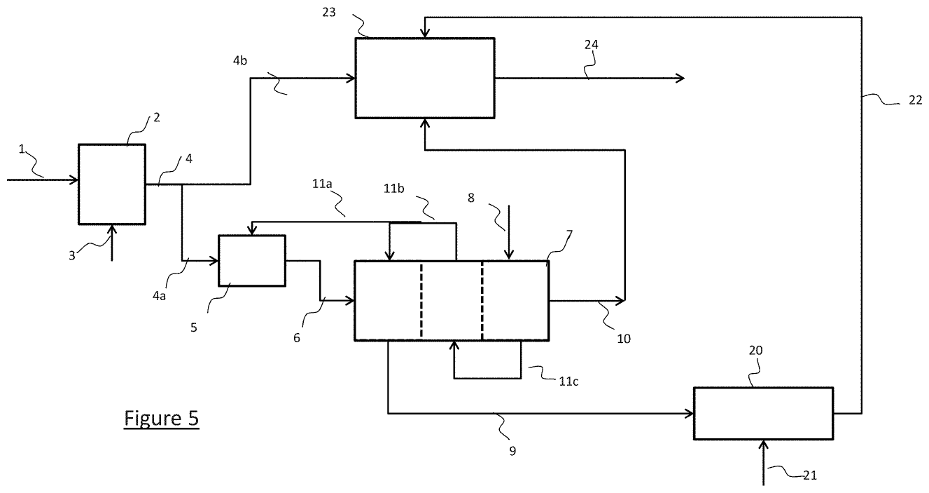

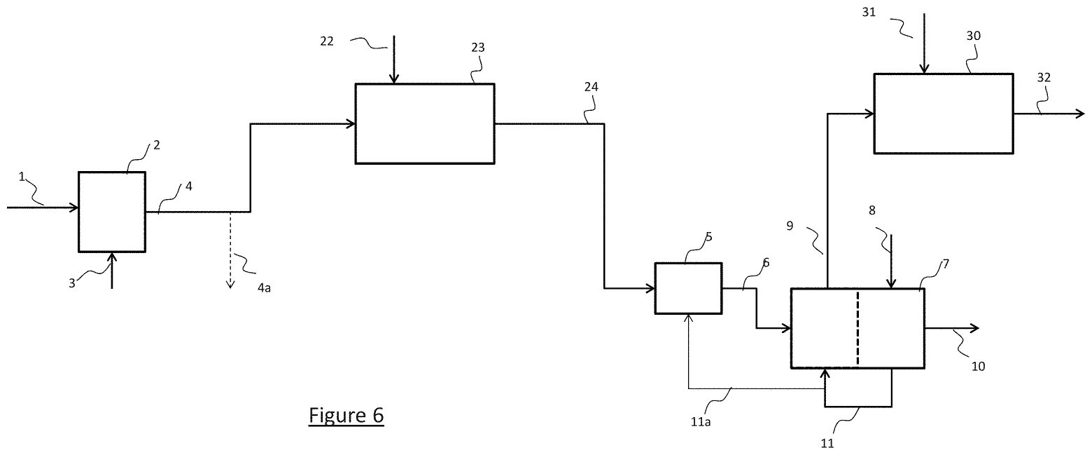

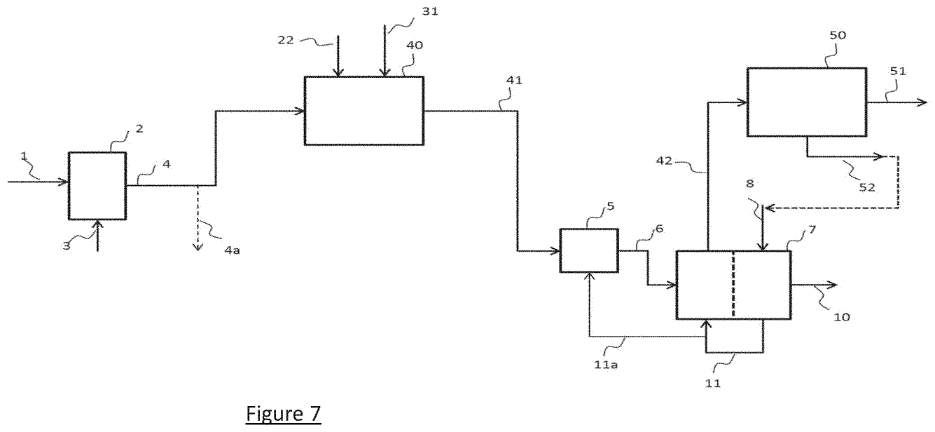

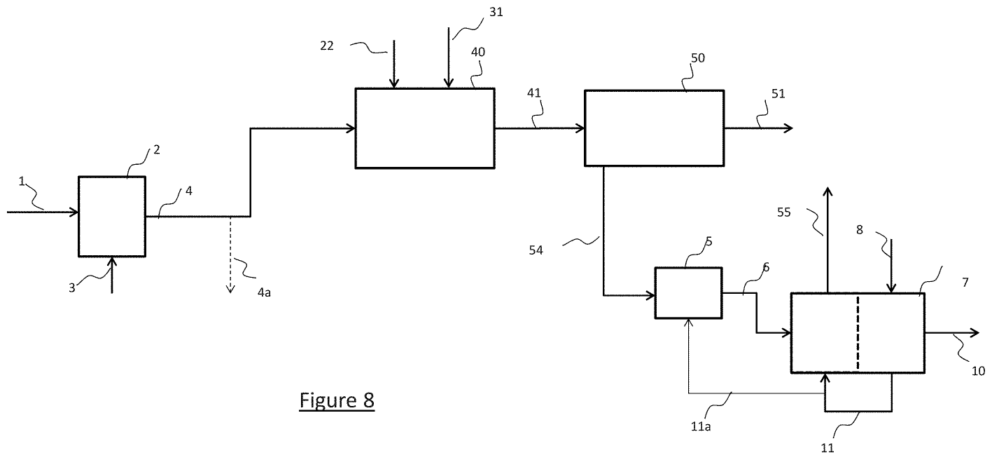

The belt filter F has in this example 10 active vacuum zones, numbered 1 to 10 in FIG. 1, where various steps of the washing and filtration process may take place (movable and resizable zones in the filter). It may be equipped with belts of polymer such as polyurethane or silicone on the filtering belt side to limit the losses of vacuum and to improve the separation. It may be envisaged to add a partition in the top part (or any other equivalent mechanical means), just after the first zone(s), to allow better distribution of the mixture on the belt.

The various streams entering and leaving these items of equipment are now described, from upstream to downstream: the pretreated marc M0 enters the metering device D of the mixer M, while the mixing fluid is introduced into the mixer via the inlets e1, e2 (shown only in FIG. 3). The mixed marc M1 (also referred to as MoRe later) leaves the downstream end of the mixer M and falls by gravity onto the belt B of the filter F in its upstream part. The belt conveys the marc to its downstream end, with extraction under vacuum, under the belt, of three liquid phases J1, J2 and J3, which are sugary liquors of decreasing concentrations. Above the belt, in its upstream part or its central part, are introduced two extraction/washing fluids f1 and f2 via nozzles spaced apart from each other. The fluid f1 may be water, the fluid f2 is the partial or total recycling of the liquor J3.

It naturally remains within the context of the invention to extract under vacuum not three liquid phases, but more, at least the same number as there are zones in the belt filter.

The movement of the extraction/washing fluid takes place counter-currentwise relative to the circulation of the mixture on the belt. In the context of the present invention, it is in fact, in a known manner, a “simulated” counter-current, in the sense that there is no actual counter-current flow in each of the washing zones. The principle of the belt filter is based on tangential washing: the mixture moves here laterally, in the figure by way of example from left to right, and the washing liquid (referred to more generally hereinabove as the extraction/washing fluid) moves from the top downwards. The injection of liquid onto the mixture which is moving on the belt may take place notably either by pouring, i.e. by gravitational flow, or by spraying. The movement from the top downwards of the liquid passing through the mixture which is moving on the belt is imposed by the vacuum that is created on the belt. The filter has sequential functioning on the mixture comprising the solid phase to be extracted (also referred to as the “cake”), which may be represented schematically as follows: —stage 1, the belt advances, —stage 2, the belt stops and a vacuum is created (and, where appropriate, pressing is performed) and liquor is extracted in each of the zones, and so on. It should be noted in general that the injection of the washing liquids is performed continuously (injection takes place when the belt is advancing and when it is at rest). The counter-current is “simulated” by reinjection of the extracted liquor upstream of its extraction.

All or a portion of at least one of the liquors J1, J2, J3 may be recycled as mixing fluid into the mixer M.

Under the 10 zones of the porous belt of the filter F, three liquid phases denoted J1, J2 and J3 are thus extracted under partial vacuum here, while the solid phase progressively enriched in solid, the “cake” G, continues its course up to the downstream part of the belt, from which it is extracted after having been subjected to a press P placed at the very end of the belt, which press terminates the separation. This pressing step is optional.

The washing fluid(s) f1, f2 are heated, for example to a temperature of from 30 to 85° C. before being poured onto the top of the cake conveyed by the porous belt, via heating means with which the delivery pipes are equipped.

The stream of sugary liquor(s) which is recycled either as mixing fluid into the mixer or as washing/extraction fluid into the filter is/are also heated before reintroduction into the filter or into the mixer, also via heating means with which their delivery pipes are equipped, for example to a temperature of from 30 to 85° C.

FIG. 2a represents a first way of implementing the installation of FIG. 1 to separate the pretreated marc of a Miscanthus-based biomass: the liquor J3 is entirely recycled into the most upstream inlet, the washing extraction fluid inlet f1 of the filter F, the liquor J2 is entirely recycled into the inlet e1 of the mixer M, as a top-up to an inlet e2 of mixer fluid in the form of water. All of the liquor J1 is extracted for use/upgrading outside of this separation installation.

FIG. 2b is another way of implementing the installation of FIG. 1 to separate the pretreated marc of a straw-based biomass. The difference with FIG. 2a is that, here, a portion of the liquor J1 also is recycled as mixing fluid into the mixer with the liquor J2.

The proportion of each of the liquors J1, J2, J3 . . . extracted from the belt filter which is recycled into the mixer or into the filter may be very variable, from 0% up to 100%. It will depend on the nature of the biomass (on its solids content), on the desired water consumption, on the desired concentration of active agents (in this case of sugars), on the liquors extracted as a function of their subsequent use, in an overall biomass treatment process, according to the needs, or as independent upgrading product.

FIG. 4 represents the integration of the separation step b into a biomass pretreatment in block diagram form. The references shown have the following meaning:
1. Biomass to be treated
2. Pretreatment step
3. Stream of fluid required for the biomass pretreatment a (water, acid catalyst, steam, etc.)
4. Stream of pretreated marc, separated into two streams 4a which goes into the separation step b and 4b (which goes into the hydrolysis step as detailed later with the aid of FIG. 5)
5. Mixer used in the separation step b according to the invention
6. Mixture leaving the mixer
7. Belt filter separator used in the separation step b according to the invention
8. Washing fluid (generally water)
9. Extracted sugary liquor
10. Washed solid
11. Intermediate liquors which are returned to the belt filter and at least a portion (11a) of which is sent to the mixer

Block 2 is the first step of conditioning and pretreatment of an inlet stream of biomass 1 with one or various reagents, by placing in contact with one or more other inlet streams 3 of fluid (water, water with a chemical catalyst of acidic or basic type, oxidizing agent, steam, etc.) and optional heat treatment(s). A stream of pretreated marc 4 is obtained as outlet stream.

Block 5 thus operates substep b1 of separation b according to the invention: it is the mixer M which receives at the inlet a portion 4a of the stream of pretreated marc 4 and at least one stream of mixing liquid comprising a portion of the liquors 11a extracted from the belt filter 7 downstream of the mixer 5.

Block 7 operates the separation substep b2 with the belt filter: it receives at the inlet the stream of solid 6 which leaves the mixer from block 5 and at least one liquid washing stream 8 with recycling of a portion 11c of the liquors extracted from downstream towards the upstream of the filter and of another portion 11a towards the inlet of the mixer of block 5, a stream of washed solid 10 leaves therefrom.

FIG. 5 represents the incorporation of the separation step b according to the invention into the whole biomass treatment process according to a first variant in accordance with FIG. 4. After blocks 2, 5 and 7 already described, the new references have the following meaning:
20. Biocatalyst production step(s) which uses the sugary liquor 9
21. Stream of fluids required for the production of biocatalysts
22. Solution containing the biocatalysts (enzymes and/or yeasts), and optionally other fluids not shown
23. Enzymatic hydrolysis step into which is introduced the other portion 4b of the pretreated marc 4 and optionally the washed marc 10 (or step of simultaneous enzymatic hydrolysis and fermentation SSF)
24. Mixture of non-hydrolysed solid and of sugars derived from the hydrolysis as a solution (or mixture of non-hydrolysed solid and of fermentation product in the case of SSF)

This figure thus shows the invention this time incorporated into the treatment process, which, beyond the pretreatment performed on the biomass, is continued by the enzymatic hydrolysis and fermentation of the marc.

The pretreated marc 4 was split into a stream 4a which is separated according to the invention as described in the preceding figure, and a stream 4b which is directed towards the enzymatic hydrolysis step 23. Optionally, the marc separated and washed 10 according to the invention may also be directed towards this step 23 with the stream 4b. (It may otherwise be upgraded differently, in the biomass treatment process or elsewhere). A stream 24 leaves therefrom, which may, depending on whether the hydrolysis takes place simultaneously with the fermentation (SSF) or not, be composed of a mixture of non-hydrolysed solid and of sugars derived from the hydrolysis (without SSF) or of a mixture of non-hydrolysed solid and of fermentation products (with SSF).

Block 20 is the step(s) of production of the biocatalysts (enzymes, yeasts) which use(s) the sugary liquor 9 derived from the separation by the belt filter of block 7 and one or more fluid streams 21 required for the production of biocatalysts (water, nutrient, other sugars, chemical products, for example for regulating the pH, etc.), this sugary liquor 9 then serving as growth/propagation substrate or as production substrate for microorganisms (fungi) producing enzymes and/or for yeasts.

In this first variant, the extracted liquor 9 is entirely used for the production of yeasts and/or enzymes. It is also possible to use it for these purposes for only a portion of the stream 9. It is also possible to upgrade all or part of this sugary liquor 9 independently of the biomass treatment process (for example upgrading it per se).

It is also possible to subject all of the pretreated marc to the separation operation according to the invention, and not only a portion thereof, and then to send all of the separated marc into the following enzymatic hydrolysis/fermentation block.

It is also possible to use directly a portion of the raw marc (i.e. of the marc at the outlet from the pretreatment step) for enzyme production and/or for yeast propagation. For the propagation of yeasts using a marc derived from the pretreatment of biomass, reference may be made, for example, to patent WO 2016/193576. For the production of enzymes using a pretreated marc, reference may be made, for example, to patent WO 2017/174378.

A stream 22 exits from block 20 containing biocatalysts (enzymes and/or yeasts), and optionally other fluids (for example: water, chemical products notably for regulating the pH, etc.), biocatalysts if produced other than on sugary liquor, nutrients, etc.), this exiting stream 22 being injected into the inlet of the enzymatic hydrolysis block 23.

Once the enzyme production has been performed, either the entire must is exploited, i.e. both the enzymes and the fungi which have produced them are exploited, or only the enzymes are exploited (in which case a prior separation/purification step must be done), to perform the enzymatic hydrolysis of the pretreated biomass.

Preferably, the enzymatic cocktail was produced by a fungus Trichoderma reesei.

As regards the production of yeasts, the yeast produced is preferably a yeast such as Saccharomyces cerevisiae which has been genetically modified to consume xylose. The stream obtained which will be exploited in the fermentation step may contain the yeasts as a whole or concentrated must; in the latter case it is referred to as a yeast cream (a prior concentration step must then be performed).

FIG. 6 proposes to incorporate the separation step b of the invention into a second variant. The new references have the following meaning:
30. Fermentation step, for example to ethanol
31. Stream of fluids required for the fermentation
32. Solution containing the fermentation product

In this instance, there is thus a block 30 for the fermentation step different from the enzymatic hydrolysis step 23, and the separation of blocks 5, 7 of the invention is inserted between these two steps: Stream 24 derived from the enzymatic hydrolysis step is separated according to the invention, and stream 9 leaving block 7 performing the separation with the belt filter is sent as an inlet stream to the fermentation block 30, which is also fed with inlet stream 31 of the fluid(s) necessary for the fermentation, such as biocatalysts (yeasts), and optionally nutrients, etc.

Stream 32 exiting the fermentation step contains the fermentation product, which can then undergo conventional separation steps, such as distillation, dehydration, solid/liquid separation (not shown) which make it possible to obtain the desired biobased molecule (in this instance ethanol as biofuel), solid residues and liquid residues also known as vinasses.

FIG. 7 proposes to incorporate the separation step b of the invention into a third variant. The new references have the following meaning:
40. hydrolysis and fermentation steps
41. Stream containing the non-hydrolysed solid and the fermentation product
42. liquid solution stream containing the fermentation product
50. Fermentation product separation step
51. Purified fermentation product stream
52. vinasse stream

Block 40 thus represents the enzymatic hydrolysis step and the fermentation step which may be separate or simultaneous, represented by a common block for the sake of clarity. Stream 41 derived from these two steps and which thus contains a portion of non-hydrolysed solid and the fermentation product is conveyed to the inlet of the separation blocks 5, 7 according to the invention. The liquid stream containing fermentation products leaving 42 from this separation feeds block 50 of the fermentation product separation step (this liquid stream thus has a different composition from stream 9 of the preceding variants, which was only a sugary liquor). At the outlet of block 50, a liquid stream of partially purified fermentation product 51 is recovered, and a vinasse stream 52 is recovered, which may optionally (dashed arrows in the figure) be recycled as replacement for the washing water 8 of the belt filter of block 7.

FIG. 8 proposes to incorporate the separation step b of the invention into a fourth variant. The new references have the following meaning:
54. medium separated from the product containing the non-hydrolysed solid and liquid
55. vinasses

In this case, the separation step b of the invention is performed after the separation step 50: stream 54 derived from the separation 50 and including a medium separated from the product containing the non-hydrolysed solid and liquid is sent into the mixer 5, the solid stream 55 leaving the belt filter of block 7 is thus vinasses extracted by washing. In this variant, the separation step 50 may be coupled to the fermentation step, which is referred to as fermentation/separation coupling, either in situ, in the fermentation reactor itself, or on another separate circuit, where the medium after separation returns in fermentation.

In all these variants, it is seen that the separation step b according to the invention can be inserted between known steps of a biomass treatment process, in this case to exploit the sugary liquors as substrates for the propagation, growth or production of enzymes of microorganisms necessary for the conversion of biomass, but also when it is desired to upgrade these sugary liquors per se.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding application No. FR 1855789, filed Jun. 27, 2018 are incorporated by reference herein.

### IMPLEMENTATION EXAMPLES

These examples follow the process schemes and the equipment detailed with the aid of the preceding figures.

Three examples will be described, on five different types of biomass: example 1 on SRC (short-rotation coppice), example 2 on Miscanthus, and example 3 on straw, Example 4 on another straw configuration, and Example 5 on another configuration on SRC.

The object is to obtain sugary liquors J1 with a minimum sugar concentration of 50 g/kg.

In the examples described below, the abbreviation “DM” denotes the dry matter content which is measured according to the standard ASTM E1756-08(2015) “Standard Test Method for Determination of Total Solids in Biomass”.

The operating conditions are as follows:

The mixer M is a Parimix IMR continuous mixer, which allows repulping of marcs with high contents of dry matter (DM) and solids. It is heated by electric means to about 40-50° C.

Separation on the belt filter F, such as the BFR continuous belt filter from the company BHS with counter-current washing, makes it possible to extract liquors concentrated in sugars. The band filter technology is based on vacuum filtration. The principle is to spread the pretreated marc sequentially over an advancing belt. This porous belt makes it possible, by vacuum suction, to separate the liquids (the liquors) from the solid, forming a cake (the washed marc), which falls at the end of the belt. The liquors are partially recycled.

The filter F has 10 active vacuum zones, where various steps of the washing and filtration process may take place (movable zones in the filter). The zones are pooled in three groups of zones, which are modulable, and which make it possible to extract three liquors J1, J2 and J3. The filter is equipped, on the sides of the porous belt, with polyurethane or silicone belts, to limit the losses of vacuum observed and to improve the filtration of the product. The filter is equipped with a vertical wall above the belt just after the mixture feed zone to allow better distribution of the mixture on the filter.

To increase the recovery of the soluble sugars and to reduce the filtration time, the recycled liquor streams J2 (and, where appropriate, J1), J3 and the washing water F1 are heated:

Liquor J3 is heated and recycled into the first washing water nozzle f1
The hot washing water was fed via the second washing nozzle f2
Liquor J2 is heated and recycled up to the repulping of the pretreated marc at the inlet of the mixer (and, where appropriate, the liquor J1).

For straw (examples 3 and 4), and for example 5 using poplarTCR, a portion of the liquor J1 was also recycled into the mixer M to increase the sugar concentration in the liquor J1.

The characteristics of the filter and the operating conditions tested are as follows:
Reference: BF025-020,
Gauze: 058000-W120 (Polypropylene 120 μm; 05-8000-S120)
Material: polymer
Flow rate: 180 kg/hour of treatable suspension
Filtration surface area: 0.5 m²
Vacuum level: between −0.5 and −1 bar

Table 1 below collates the solids content and dry matter DM content characteristics of the feedstock (pretreated biomass M0) which is separated according to the invention:

TABLE 1

| | Example 5: | Example 4: | Example 3: | Example 2: | Example 1: |
|---|---|---|---|---|---|
| | SRC (2) | Straw (2) | Straw | Miscanthus | SRC |
| Pretreated | 44% solid | 29-32% solid | 29-30% solide | 42% solid | 38-43% solid |
| Biomass M0 | 53% MS | 41-46% MS | 42-44% MS | 54% MS | 49-53% MS |

The rotation speed of the feed screw of the mixer M, and thus the flow rate of the pretreated marc M0, is defined by the frequency of the metering device D. This speed has an influence on the mixing and the consistency of the repulped marc leaving the mixer. An excessively high mixing screw speed results in a repulped marc that is too viscous for the rest of the process. A weighing hopper is generally provided on the installation (not shown) along with a means for measuring the flow rate of marc, and the feed can be regulated by slaving the screw speed to this flow rate measurement. It may also be chosen to operate with a set screw speed, which will have been calibrated beforehand.

Table 2 below presents the flow rates of raw marc and of recycled liquor entering the mixer:

TABLE 2

| | Flow rate of raw marc | recycled liquor |
|---|---|---|
| | inlet (kg/h) | (kg/h) |
| SRC (2) (example 5) | 50 | 130 |
| Straw (2) (example 4) | 50 | 130 |
| Straw (example 3) | 50 | 129 |
| Miscanthus (example 2) | 70 | 95 |
| SCR (example 1) | 50 | 75 |

### Example 1 According to the Invention: Biomass Based on Short-Rotation Coppice (SRC)

The tests began with an SRC, which is often considered as a raw material that is difficult to process. To improve the extraction, spraying nozzles were installed, a vacuum of −0.6 bar was overall material balance and the operating conditions are detailed in table 3 above.

TABLE 3

| | Units | SRC(2) | Straw (2) | SRC | Straw | Miscanthus |
|---|---|---|---|---|---|---|
| Flow rate M0 | kg/h | 50 | 50 | 50 | 50 | 70 |
| Flow rate recycled J2 | kg/h | 110 | 110 | 75 | 110 | 90 |
| Flow rate recycled J1 | kg/h | 20 | 20 | 0 | 20 | 0 |
| Flow rate at outlet | kg/h | 180 | 180 | 125 | 180 | 160 |
| of the mixer M | | | | | | |
| Feed time | s | 19 | 17 | 19 | 17 | 15 |
| Vacuum pressure | bar | −0.6 | −0.45 | −0.6 | −0.45 | −0.15 |
| Pressing pressure | bar | — | — | 3.3 | 3.7 | 2.6 |
| Temperature J1) | ° C. | 40 | 45 | 39 | 45 | 32 |
| Flow rate | kg/h | 67 | 50 | 45 | 70 | 55 |
| Temperature J2 | ° C. | 80 | 80 | 75 | 75 | 75 |
| after exchanger | | | | | | |
| Flow rate J2 | kg/h | 75 | 110 | 75 | 115 | 90 |
| produced | | | | | | |
| Temperature J3 | ° C. | 80 | 80 | 75 | 75 | 75 |
| after exchanger | | | | | | |
| Flow rate J3 | kg/h | 75 | 105 | 60 | 105 | 80 |
| Flow rate of | kg/h | 70 | 69 | 55 | 83 | 55 |
| washing water | | | | | | |
| Température | ° C. | 50 | 50 | 50 | 50 | 50 |
| Temperature of | | | | | | |
| washing water | | | | | | |
| Temperature M1 | ° C. | 26 | 35 | 40 | 35 | 45 |
| Flow rate M1 | kg/h | 60 | 65 | 60 | 52 | 92 |

### Example 2 According to the Invention: Biomass Based on Miscanthus

The extraction of the liquor on the pretreated marc obtained from Miscanthus posed no problems. The washing (also referred to as mixing, performed by the mixer M) is very efficient and liquors with a sugar concentration of between 58-60 g/kg were produced. The overall material balance and the operating conditions are detailed in the preceding table, and in table 4 below.

The belt filter F was fed with 160 kg/h of MoRe (18% DM) at 58° C. Two overflow washing nozzles were installed on zones 4 and 6 of the 10 active zones of the belt. To have the greatest possible space between the streams, the first nozzle washed counter-currentwise relative to the direction of advance of the filter, and the second co-currentwise. 45 kg/h of liquor J1 at 65-68 g/kg of sugars were produced in the first two days. Next, on the third day, the washing water flow rate was increased to produce more liquor with a slightly lower concentration. Consequently, 55 kg/h of J1 were produced with a sugar concentration of between 58 and 60 g/kg.

### Example 3 According to the Invention: Biomass Based on Straw

The first test on straw was performed with the same configuration as for the two preceding examples. Consequently, liquor J1 has a low sugar concentration, of 38.77 g/kg. To increase it, recycling of J1 (20 kg/h) into the J2 tank was established and the mixture of the liquors J1 and J2 was heated and recycled into the mixer M. To improve the vacuum and close the cake which had a tendency to crack, the first washing nozzle was by spraying (zone 4) and the second by overflow (zone 6). Specifically, 70 kg/h of liquor J1 at 53-57 g/kg of sugars were produced. The operating conditions are detailed in the preceding table 4.

### Example 4 According to the Invention: Straw Biomass

This test has the same configuration as in Example 3, also with straw-based biomass, therefore, with the following differences: here the final press (pressing the washed mare cake at zone 9 of the belt filter) is not used, the first washing nozzle is used by pouring and non-spraying, and the second washing nozzle is used by spraying and not pouring, Juice J1 produced has a higher concentration of sugars (50-66 g/kg of sugar). The operating conditions are detailed in Table 4 below.

### Example 5 According to the Invention: Biomass Based on SRC

This test has the same configuration as Example 1, also with a SRC-based biomass, with the following differences: here, the final press (pressing the washed marc cake at zone 9 of the band filter) is not used, and the first wash nozzle is used by pouring and not spraying. The juice J1 produced has a concentration of 51 g/kg of sugar. The operating conditions are detailed in Table 4 below.

To rapidly check on site if the sugar concentration in liquor J1 indeed reached 50 g/kg as demanded, measurements were taken with an Atago brand PAL-1 refractometer.

Table 4 below presents the feedstock characteristics: a mixture of M0 with liquors J2 (or J1+J2 for straw) and the products J1 and M0 and quantifies the efficiency of the separation obtained with the invention: the separation of straw was most efficient (99%).

TABLE 4

| | | Ex 5: | Ex 4: | Ex 3: | Ex 2: | Ex 1: |
|---|---|---|---|---|---|---|
| | | SRC (2) | Straw(2) | Straw | Miscanthus | SRC |
| Feedstock | Pretreated | 44% solids | 29-32% solids | 29-30% solids | 42% solids | 38-43% solids |
| | biomass M0 | 53% DM | 41-46% DM | 42-44% DM | 54% DM | 49-53% DM |
| | | 80 g/kg | 65-93 g/kg | ~90 g/kg | ~95 g/kg | ~83 g/kg |
| | | of sugar | of sugar | of sugar | of sugar | of sugar |
| | Recycled | ~0% solids | ~0% solids | ~0.003% solids | ~0% solids | ~0% solids |
| | liquor J2 | 4% DM | 5-11% DM | 6% DM | 4% DM | 8.6% DM |
| | | 23 g/kg | 22-37 g/kg | ~33 g/kg | ~27 g/kg | ~50 g/kg |
| | | of sugar | of sugar | of sugar | of sugar | of sugar |
| | Pretreated | 12% solids | 8.3% solids | 8.5% solids | 18% solids | 16% solids |
| | biomass | 18% DM | 18% DM | 16% DM | 26% DM | 25% DM |
| | M0Re | | | | | |
| Product | J1 | ~0.04% solids | ~0.04% solids | ~0.03% solids | ~0.09% solids | ~0.07% solids |
| | | 8% DM | 10-11% DM | 9-10% DM | 9-10% DM | 11% DM |
| | | 51 g/kg | 50-66 g/kg | 53-57 g/kg | 5-60 g/kg | 69-70 g/kg |
| | | of sugar | of sugar | of sugar | of sugar | of sugar |
| | M1 | 38% solids | 23-26% solids | 30-32% solids | 31-33% solids | 33-34% solids |
| | | 40% DM | 28-30% DM | 32-33% DM | 37-38% DM | 35-37% DM |
| | | 26 g/kg | 19-36 g/kg | 0.3-1 g/kg | 32-38 g/kg | 13-20 g/kg |
| | | of sugar | of sugar | of sugar | of sugar | of sugar |
| Separation | E | ~85% | ~71-77% | 99% | ~62% | ~79% |
| efficiency | | | | | | |

In conclusion, it is seen that these five examples according to the invention, with different initial biomasses, managed to achieve the objective of an extracted liquor J1 with the ofsired sugar concentration, largely above the set minimum threshold of 50 g/kg. The separation according to the invention with the two lines of equipment in series, continuous mixer and continuous (belt) filter, allows a lot of flexibility and great compactness. They can thus be inserted without difficulty into a biomass treatment installation at various steps, as soon as there is a need to perform a solid/liquid separation on a mixture with a ofsire to upgraof both the solid part and the liquid part obtained after separation.

The invention thus makes it possible, irrespective of the pretreated biomass and its rheological constraints, to obtain a sugary liquor with a concentration of greater than 50 g/kg, which is not the case when the invention is not performed, as shown in the following comparative examples:

### Comparative Examples (not in Accordance with the Invention)

The pretreated biomasses of two of the preceding examples are used in a configuration not in accordance with the invention: the substrate M0 is mixed in a mixer M with water and then separated on the belt filter F. In this implementation not in accordance with the invention, there is no recycling of a stream extracted from the belt filter F to the mixer M.

### Comparative Example 6: Biomass Based on SRC

The SRC pretreated biomass M0 ofrived from Example 1 is mixed in a mixer M with water and then separated on the belt filter F, which is itself operated according to the configuration of Example 1. Owing to its rheology, the SRC pretreated biomass must be diluted to a solids content of less than 16% to form a cake that is able to be filtered on the belt filter F. The composition of the SRC marc M0 of Example 1 is recalled: solids content of 38%, sugar content of 83 g/kg. To obtain a correct rheology at the mixer outlet, it is necessary to lower the solids content of the substrate after mixing: it is thus necessary to add 68.8 kg of water to 50 kg of marc M0 into the mixer, and this mixture is then ofposited on the belt filter. The first zone of the belt filter allows the production of a concentrated liquor J1 with a sugar content of only 41.6 g/kg. In this example not in accordance with the invention, it is seen that it is not possible to achieve a concentration of 50 g/kg of sugars in liquor J1 for the SRC substrate.

### Comparative Example 7: Biomass Based on Straw

The straw pretreated biomass M0 ofrived from Example 3 is mixed in a mixer M with water and then separated on the belt filter F, which is itself operated according to the configuration of Example 3. Owing to its rheology, the straw pretreated biomass must be diluted to a solids content of less than 9% to form a cake that is able to be filtered on the belt filter F. The composition of the straw marc M0 of Example 3 is recalled: solids content of 30%, sugar content of 90 g/kg. To obtain a correct rheology at the mixer outlet, it is necessary to lower the solids content of the substrate after mixing: it is thus necessary to add 117 kg of water to 50 kg of marc M0 into the mixer, and this mixture is then ofposited on the belt filter. The first zone of the belt filter allows the production of a concentrated liquor J1 with a sugar content of 27 g/kg. In this example not in accordance with the invention, it is seen that it is not possible to achieve a concentration of 50 g/kg of sugars in liquor J1 for the straw substrate.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for treating a lignocellulosic biomass, said process comprising at least the following step:
a. Pretreatment of the biomass via at least one cooking or steam explosion operation, so as to obtain a pretreated substrate,
characterized in that it also comprises at least one step of:
b. Liquid/solid separation on at least a portion of the pretreated substrate obtained on conclusion of step a or on conclusion of an additional step of treatment of said substrate after step a, said separation step comprising two successive substeps:
an upstream substep b1 of contacting the solid/liquid mixture performed with a continuous mixer (M) using a mixing fluid, optionally preceofd by a pump, and
a downstream sub-step b2 of extraction/washing performed with a continuous filter, notably a belt filter (F), using a washing fluid, to obtain a solid phase enriched in solid and a plurality of liquid phases enriched in liquid, with at least partial recycling of a liquid phase extracted from the filter at the inlet of the mixer as a mixing fluid.

2. Process according to claim 1, characterized in that the continuous filter functions counter-currentwise between the circulation of the solid/liquid mixture to be separated and the extraction/washing fluid.

3. Process according to claim 1, characterized in that the separation of the solid/liquid mixture is performed during step b on a first portion of the pretreated substrate obtained on conclusion of step a, in that a second portion of said pretreated substrate is subjected to at least one subsequent treatment step, notably hydrolysis, and in that the solid phase enriched in solid obtained in step b is also subjected to at least one subsequent treatment step, notably the same as those to which the second portion of the pretreated substrate is subjected.

4. Process according to claim 1, characterized in that it comprises a step e of enzymatic hydrolysis of the pretreated substrate ofrived from the pre-treatment step a to obtain a hydrolysate, in that the solid/liquid separation b is performed on at least a portion of said hydrolysate.

5. Process according to claim 1, characterized in that the solid/liquid separation b is performed on at least a portion, notably all, of the pretreated substrate obtained on conclusion of the pretreatment step a to obtain a solid phase enriched in solid, and in that it comprises a step e of enzymatic hydrolysis of said solid phase enriched in solid to obtain a hydrolysate.

6. Process according to claim 1, characterized in that the liquid phases enriched in liquid ofrived from the separation step b are sugary liquors, and in that said process also comprises a step c of producing enzymes and/or a step d of producing yeasts, and in that at least one of said sugary liquors is used for said production of enzymes c or of yeasts d.

7. Process according to claim 1, characterized in that it also comprises the following steps after the pretreatment step a:
e. Enzymatic hydrolysis of the pretreated substrate, so as to obtain a hydrolysate including a solid material and a liquid phase containing sugars,
f. Fermentation of the hydrolysate, so as to obtain a fermentation wine,
the fermentation step f possibly succeeding or being concomitant with the enzymatic hydrolysis step e,
g. Separation/Distillation of the fermentation wine, so as to obtain a distillation product in the form of an alcohol, a solvent or another biobased molecule, and vinasses,
step b being performed on at least a portion of the products obtained after the hydrolysis step e and/or fermentation step f and/or separation/distillation step g.

8. Process according to claim 1, characterized in that, in the separation step b, the extraction/washing fluid is heated before introduction into the filter (F) and/or the liquid phase extracted from the filter is recycled before its introduction into the mixer, notably to a temperature of at least 30° C., notably of not more than 90° C., notably a temperature of between 40° C. and 80° C.

9. Process according to claim 1, characterized in that, in step b, the filter is a belt filter (F) which comprises at least two, notably at least three, successive zones, with withdrawal of a liquid phase in each zone.

10. Process according to claim 1, characterized in that the liquid phase from the first and/or the second zone is recycled at least partly into the inlet of the mixer (M) of step b, and in that the liquid phase withdrawn from the third and/or the last zone is at least partly recycled as washing/extraction fluid for the belt filter (F).

11. Process according to claim 1, characterized in that the operating parameters of the separation steps b1 and b2 may be selected as a function of the characteristics of the solid/liquid mixture, so that the liquid phases produced extracted from the belt filter (F) have a concentration of at least 50 g of product/kg, notably at least 50 g of sugar/kg when the separation b is performed on the pretreated substrate obtained from the pre-treatment step a.

12. Installation for treating a lignocellulosic biomass, characterized in that it comprises at least:
one zone for pretreatment of the biomass via at least one cooking or steam explosion operation, so as to obtain a pretreated substrate,
one zone for liquid/solid separation on at least a portion of the pretreated substrate obtained at the outlet of the pretreatment zone or of an additional treatment zone of said substrate after the pretreatment zone, said separation zone comprising two subzones in series: —one upstream contacting subzone comprising a continuous mixer (M) using a mixing fluid, optionally associated with an upstream pump, and—one downstream extraction/washing subzone comprising a continuous filter, notably a belt filter (F), with a washing fluid, the filter preferably functioning counter-currentwise between the circulation of the solid/liquid mixture to be separated and the extraction/washing fluid, and at least partial recycling of a liquid phase extracted by the filter into the inlet of the mixer as a mixing fluid.

13. Treatment installation according to claim 1, characterized in that it envisages, in the separation zone: —fluid connection means between the filter (F) and the mixer (M) to recycle at least a portion of a liquid phase extracted by the filter into the inlet of the mixer and—fluid connection means for recycling another liquid phase extracted from the filter (F) as a washing fluid for said filter.

14. Treatment installation according to claim 12, characterized in that it also comprises an enzyme production zone and/or a yeast production zone, and in that it envisages a means for transferring at least one liquid phase extracted from the belt filter from the separation zone to the enzyme production zone and/or to the yeast production zone, said liquid phase being a sugary liquor.

15. The process according to claim 1 wherein said biomass is selected from wood, straw, agricultural residues, and all dedicate energy crops, notably annual or perennial plants such as miscanthus in order to produce sugars, biofuels or biobased molecules.
